(19) United States
(12) Patent Application Publication
(10) Pub. No.: US 2017/0056543 A1
(43) Pub. Date: Mar. 2, 2017

(54) AIR DECONTAMINATION DEVICE & METHOD

(71) Applicant: LORRAINE INGRID BALDRY, LONDON (GB)

(72) Inventor: Alan Mole, London (GB)

(21) Appl. No.: 15/118,663

(22) PCT Filed: Feb. 10, 2015
(86) PCT No.: PCT/GB2015/050362

§ 371 Date: Aug. 12, 2016

(30) Foreign Application Priority Data
Feb. 14, 2014 (GB) 1402624.9

(51) Int. Cl. A61L 9/22; B01D 53/86; B01D 53/32; A61L 9/04; A61L 9/12

(52) U.S. Cl. CPC A61L 9/22; A61L 9/04; A61L 9/12; B01D 53/32; B01D 53/8675; A61L 2209/11; B01D 2259/804; B01D 2259/818; B01D 2255/802; B01D 2257/106

(57) ABSTRACT

A device for air decontamination comprises a housing (10) having an air inlet (14), an air outlet (16) and an air flow passage (12) therebetween, the housing including at least one non-thermal plasma cell (22); wherein the non-thermal plasma cell is sized and positioned relative to the internal dimensions of the housing such that a portion of air entering the housing from the air inlet is adapted to pass through and across the non-thermal plasma cell and a portion of air entering the housing from the air inlet is adapted to pass outside of the external surface of the non-thermal plasma cell. A method of decontaminating air is also disclosed.

The present invention relates to an improved device and method for the decontamination of air.

The present inventor has been working in the field of air treatment for many years, notably obtaining a European patent for an air decontamination device and method under number EP 1799330 B1, the contents of which are hereby incorporated by reference.

This earlier decontamination device comprises a non-thermal plasma filter, an ultraviolet radiation emitting device, an ozone catalysing device, a hydrocarbon emitter, and an air stream generator by which an air stream can be generated and directed to pass through or across the non-thermal plasma filter, the UV radiation emitting device, the ozone catalysing device and the hydrocarbon emitter.

The method relating to this earlier decontamination device comprises the steps of:

a) directing an air stream to be decontaminated through a non-thermal plasma filter so that free radicals are produced by which contaminants in the air stream are neutralised;

b) breaking down ozone in the air stream output from the non-thermal plasma filter and increasing a level of said free radicals; and

c) introducing a hydrocarbon having a carbon-carbon double bond into the air stream to preferentially react with residual ozone and cause a free radical cascade, the air stream becoming suitable for human exposure.

This earlier invention seeks to increase the level of hydroxyl radicals in an indoor environment, since hydroxyl radicals are plentiful in outside air and are known to be effective in reducing the level of air contaminants, including pathogenic bacteria and viruses. At the same time, consideration needs to be given to elimination, from the treated air, of any unwanted by-products of the method, such as ozone or formaldehyde.

The present invention seeks to provide an improved device for the decontamination of air. Whilst the earlier device is effective in providing air which has been decontaminated and is suitable for human exposure, the inventor has sought to improve its performance, for example in terms of efficiency of decontamination, energy usage and noise levels. In particular the inventor has sought to achieve an effective output of active radicals with the minimum levels of ozone and hydrocarbon, to meet the strictest international Indoor Air Quality standard. This development work has resulted in discoveries which form the basis of the present invention.

According to the present invention there is provided a device for air decontamination comprising a housing having an air inlet, an air outlet and an air flow passage therebetween, the housing including at least one non-thermal plasma cell located downstream of the air inlet; wherein the plasma cell is sized and positioned relative to the internal dimensions of the housing such that a portion of the air entering the housing from the air inlet is adapted to pass through and across the plasma cell and a portion of the air entering the housing from the air inlet is adapted to pass outside of the external surface of the plasma cell.

The device of the present invention may be a stand-alone and/or portable unit; it may be mounted on a stand or on a surface. It is preferably configured to decontaminate air within a room, for example, rather than being configured to form part of a heating, ventilating and/or air conditioning system. The device may be used in any desired orientation, including a horizontal or a vertical orientation. The housing of the device may have the general shape of a cylinder or of a rectangular prism.

The non-thermal plasma cell is preferably sized such that it does not extend to all or some of the internal surface wall(s) of the housing, thereby defining at least one channel or passage between the external surface of the plasma cell and the internal surface wall(s) of the housing. Beyond the non-thermal plasma cell, the channel or passage continues in the direction of the length of the housing, although it may not reach the air outlet of the device. The non-thermal plasma cell is preferably substantially centrally-positioned within the width of the housing.

The non-thermal plasma cell, in one embodiment, comprises: an annulus of a dielectric material formed from a continuous wall of material having apertures therein; and a pair of annular air-permeable electrodes mounted on opposing sides of the wall of the dielectric. Such a non-thermal plasma cell is described in the inventor's UK patent application GB2496888 A, the contents of which are hereby incorporated by reference.

The plasma cell is preferably electronically controlled so that emissions of residual ozone from the air decontamination device lie between defined parameters in relation to the volume of air emitted by the device, for example not less than 5 ppb (parts-per-billion) and not more than 45 ppb in an air stream of 25 m³/hr. In one example, the emissions of residual ozone from the air decontamination device of the present invention are about 5 ppb in an air stream of 25 m³/hr.

The present invention is safe and effective in very small chambers. Undesirable by-products have been reduced from the output.

During use of the device, air flows therethrough. Preferably no use is made of a flow diverter, such as a pivotally-mounted gate, to direct a portion of air into a by-pass duct, for example. Preferably, the device is not adapted to be isolated from the surrounding environment in terms of air flow therethrough.

In the earlier devices and methods of the inventor, a stream of air is generated such that air flows across the surface of the outer electrode of the plasma cell and then through the voids in the dielectric and inner electrode. No portion of the air is adapted to pass outside of the external surface of the plasma cell: there is no single air flow passage and there are no multiple air flow passages permitting air to pass over the external surface of the non-thermal plasma cell and over the external surface of the ozone catalysing device. Instead, the generated air stream is directed to pass through or across the non-thermal plasma filter. In one example, the plasma cell extends across the complete diameter of a cylindrical air decontamination device, and the air stream is directed to pass through the plasma cell accordingly. Free radicals are thereby generated within the air stream, including O. (oxyl) and OH. (hydroxyl). These free radicals are powerful oxidants and will oxidise hydrocarbons, organic gases and particles, typically PM 2.5 (being particulate matter up to 2.5 micrometers in size) and below, such as bacteria, viruses, spores, yeast moulds, malodours and small carbon particles which are directly linked to cardio-pulmonary diseases.

The present inventor has made the surprising discovery that free radicals can be generated within air that does not flow across the surface of the outer electrode of the plasma cell and then through the voids in its dielectric and inner electrode, so long as this air flows in the general vicinity of the plasma cell. This is because the plasma field produced by the plasma cell has been found to be greater than the dimensions of the plasma cell itself. Thus, according to the present invention, air flowing into the device for air decontamination does not have to pass through and across the plasma cell, as it did in the earlier devices.

According to a preferred embodiment of the present invention, the air flow passage in the housing of the air decontamination device comprises a first air passage and a second air passage, the first air passage being adapted to carry the portion of air which passes through and across the plasma cell and the second air passage being adapted to carry the portion of air which passes outside of the external surface of the plasma cell. Both the first and the second air passages may extend in the direction of the length of the housing beyond the non-thermal plasma cell, along the air flow passage.

Air flowing in the second air passage passes sufficiently close to the external surface of the plasma cell for free radicals to be generated therein. Preferably, the first air passage and the second air passage are permeable to permit air to move therebetween.

The air decontamination device preferably comprises at least one UV radiation emitting device and/or at least one ozone catalysing device which are positioned within the housing, coincident with, partly coincident with or downstream of the plasma cell. These devices act to control ozone in the air stream output from the non-thermal plasma cell, and to produce hydroxyl radicals to enhance the production of more oxidising radicals such as OH. (hydroxyl) and OOH. (hydroperoxyl). Preferably both a UV radiation emitting device and an ozone catalysing device are present in the housing, with the UV light incident on the ozone catalysing device. In one embodiment, the UV radiation emitting device is surrounded, at least in part, by the ozone catalysing device. The UV radiation emitting device may be disposed in the air flow passage of the housing, coincident with, partly coincident with or downstream of the plasma cell, and it may be substantially coincident with the ozone catalysing device. In a preferred embodiment, the UV radiation emitting device is positioned, at least in part, within the annulus of a non-thermal plasma cell, whereby the plasma field generated in this region by the plasma cell causes emission of the radiation without need for a separate power source for the UV radiation emitting device.

The UV radiation emitting device is preferably positioned within the first air passage of the housing. The ozone catalysing device is preferably positioned such that air in the first air passage is adapted to pass through the ozone catalysing device and air in the second air passage is adapted to pass over the external surface of the ozone catalysing device.

In one embodiment, the external surface of the first air passage is defined by the external surface of the plasma cell and the external surface of the ozone catalysing device. Preferably, the internal surface of the second air passage is defined by the external surface of the plasma cell and the external surface of the ozone catalysing device, and the external surface of the second air passage is defined by the internal surface of the housing. In this respect, the terms ‘internal’ and ‘external’ refer to the distance of the surfaces from the centre of the device, for example in an axial or width-wise direction. A bridging piece may be positioned between the plasma cell and the ozone catalysing device to assist in the formation of the first and second air passages.

Preferably, the plasma cell, the UV radiation emitting device and the ozone catalysing device all lie in a substantially straight line along the length of the housing, although this is not essential, with the ozone catalysing device preferably surrounding the UV radiation emitting device, at least in part. In one embodiment, the plasma cell, the UV radiation emitting device and the ozone catalysing device are substantially centrally-disposed within the width of the housing, with at least one channel or passage being defined for unobstructed air flow. The UV radiation emitting device may be at least partly surrounded by the non-thermal plasma cell.

In a preferred embodiment, the housing is elongate and the first and second air passages extend in the direction of the length of the housing. The first air passage may be surrounded by the second air passage. For example, the first air passage may be located within the second air passage, thereby forming inner and outer passages. Preferably the UV radiation emitting device and/or the ozone catalysing device are elongate and they also extend in the direction of the length of the housing. This assists in the formation of effective first and second air passages. This also assists in increasing the dwell time of the air adjacent the UV radiation emitting device and the ozone catalysing device, thereby assisting in the removal of by-products.

In one embodiment, the UV radiation emitting device is centrally disposed within the plasma cell and derives power directly from the field of the plasma without any electrical contact. This arrangement may be sufficiently efficient for the ozone catalysing device to be substantially shorter in length than in other embodiments.

The first and second air passages may or may not extend to the air outlet of the housing, since there may be termination of these passages short of the air outlet to facilitate mixing of their respective air flows in the housing before the air exits the housing. The first and second air passages may extend substantially along the whole length of the device, although it is possible for them to be shorter than this: preferably, the first and second air passages have a length which is more than half the length of the housing. In a preferred embodiment, the first and second air passages each have substantially consistent dimensions along their length. Preferably, the first air passage has a substantially-circular cross section in a direction which is perpendicular to the length of the housing and the second air passage is substantially-annular in cross section in a direction which is perpendicular to the length of the housing, the first air passage being positioned within the annulus of the second air passage.

By way of example, about 25 to 30% of the incoming air stream is directed into the first air passage and about 70 to 75% of the incoming air stream is directed into the second air passage. Alternatively, for high airflow models, the ratio may be varied according to operational requirements.

In one embodiment, approximately 33% of the air in the second air passage is exposed to the plasma cell at the surface of the plasma cell.

The plasma field generally extends with diminishing strength to approximately the half-way point between the inner and outer surfaces of the second air passage, in one embodiment. The distance of extension of the plasma field and the density of the plasma field may be controlled by adjustment of the plasma cell power supply.

In another embodiment, the housing further comprises a shield extending in the direction of the air flow passage, the internal surface of the shield being spaced from and facing the non-thermal plasma cell, such that a portion of air entering the housing from the air inlet is adapted to pass outside of the external surface of the shield, this portion of air being separate from the portion of air which is adapted to pass through and across the non-thermal plasma cell and being separate from the portion of air which is adapted to pass outside of the external surface of the non-thermal plasma cell. The shield is preferably positioned between, and spaced from, the non-thermal plasma cell and the wall or walls of the housing.

The housing preferably further comprises a third air passage which is adapted to carry the portion of air which passes outside of the external surface of the shield.

The shield is preferably adapted to shield air flowing in the third air passage from electromagnetic emissions from the non-thermal plasma cell. It may also provide a light-proof barrier to the UV rays from the ultraviolet (UV) radiation emitting device.

The shield may be used to form an electrostatic surface, acting as an electrostatic decontamination device for the deposition of particles in the first and second air passages charged by proximity to the plasma cell. In this respect, the shield may be earthed.

The shield is preferably impermeable to air to prevent air moving from either the first air passage or the second air passage into the third air passage.

The internal surface of the shield may be coated with a catalyst, for example titanium dioxide, to further enhance the breakdown of excess ozone, and to utilise incident UV light to produce a greater yield of hydroxyl radicals.

In one embodiment, the internal surface of the third air passage is defined by the external surface of the shield, and the external surface of the third air passage is defined by the internal surface of the housing. Preferably, the internal surface of the second air passage is defined by the external surface of the non-thermal plasma cell and the external surface of the ozone catalysing device, and the external surface of the second air passage is defined by the internal surface of the shield; and preferably, the external surface of the first air passage is defined by the external surface of the plasma cell and the external surface of the ozone catalysing device.

In a preferred embodiment, the housing is elongate and the first, second and third air passages extend in the direction of the length of the housing, with the first air passage being surrounded by the second air passage and with the second air passage being surrounded by the third air passage.

The first, second and third air passages may or may not extend to the air outlet of the housing, since there may be termination of these passages short of the air outlet to facilitate mixing of their respective air flows in the housing before the air exits the housing. Mixing of the respective air flows of the first, second and third air passages enables the air flow from the third air passage to dilute the air flow from the first and second air passages, therefore helping to emit air which has an acceptable air quality in terms of ozone content, for example.

The first, second and third air passages may extend substantially along the whole length of the device, although it is possible for them to be shorter than this: preferably, the first, second and third air passages have a length which is more than half the length of the housing. In a preferred embodiment, the first, second and third air passages each have substantially consistent dimensions along their length.

The different portions of air flowing towards the exit of the housing at the air outlet are preferably adapted to be mixed together at or adjacent to the air outlet to provide the desired dilution effect: in this respect, the untreated air from the third air passage dilutes the treated (decontaminated) air from the first and second air passages.

Preferably, the first air passage has a substantially circular cross section in a direction which is perpendicular to the length of the housing and the second air passage is substantially annular in cross section in a direction which is perpendicular to the length of the housing, the first air passage being positioned within the annulus of the second air passage. Preferably, the third air passage is substantially annular in cross section in a direction which is perpendicular to the length of the housing, the first and second air passages being positioned within the annulus of the third air passage.

In one example, about 50% of the incoming air stream is directed into the first and second air passages and about 50% of the incoming air stream is directed into the third air passage.

The shield preferably has a length extending from at least the base of the non-thermal plasma cell to at least the end of the ozone catalysing device remote from the non-thermal plasma cell and/or to at least the end of the ultraviolet (UV) radiation emitting device remote from the non-thermal plasma cell.

The air decontamination device of the invention may further comprise a device for delivering hydrocarbons in the vicinity of the air outlet of the housing of the air decontamination device. This hydrocarbon delivery device may have an air inlet, an air outlet and an air flow passage therebetween. The hydrocarbon delivery device is preferably adapted to deliver hydrocarbons by using a flow of untreated air which enters and exits the hydrocarbon delivery device, this untreated air being air which has not been decontaminated by the effects of the non-thermal plasma cell; by way of example, this untreated air is not passed through the first and second air passages of the air decontamination device.

In one embodiment, the hydrocarbon delivery device is located in the third air passage: the third air passage may provide the air flow passage between the air inlet and the air outlet of the hydrocarbon delivery device. The hydrocarbons may be delivered either by being blown by the air in the air flow passage or by using a syphoning effect.

In another embodiment, the air flow passage of the hydrocarbon delivery device is in fluid communication with the air flow passage of the air decontamination device at the air inlet and air outlet of the hydrocarbon delivery device and the air flow passage of the hydrocarbon delivery device is otherwise located outside of the housing of the air decontamination device.

The air flow passage may therefore be located, at least in part, outside of the housing of the air decontamination device. This air flow passage may only be in fluid communication with the air flow passage of the air decontamination device at the air inlet and air outlet of the hydrocarbon delivery device. The air inlet of the hydrocarbon delivery device is preferably adapted to draw air from the vicinity of the air inlet of the housing of the air decontamination device, in order to preclude polymerisation or oxidation of terpene (for example) at the release point.

The hydrocarbon delivery device preferably comprises a reservoir of one or more hydrocarbons (for example a liquid hydrocarbon) located in the air flow passage of the hydrocarbon delivery device, such that incoming air is adapted to pick up the hydrocarbons as it flows through the air flow passage. The hydrocarbons in this air are delivered within the housing of the air decontamination device in the vicinity of the air outlet to react with residual ozone to control the air quality. The resulting air stream is suitable for human exposure, whilst providing the capacity for generation of oxidising radicals in the chamber/area being treated: the hydrocarbon may interact with ozone generated by the device to initiate a chain reaction of radicals outside of the device. Preferably the level of radicals in the treated air, as measured by Laser Induced Fluorescence Spectroscopy (LIFS), is not less than 10⁶/cc air, when measured 1 metre from the air decontamination device. Preferably the concentration of hydroxyl radicals in the treated air is in the range of 1×10⁶ to 6×10⁷ per cc of air. For example, the concentration of free radicals in the treated air is in the range of 4×10⁶ to 6×10⁶ per cc of air.

The hydrocarbon preferably has at least two carbon-carbon double bonds. It is preferably a straight-chain olefin. In one embodiment the olefin is a terpene, which may be myrcene or linalool, such that the hydrocarbon is selected from the group consisting of myrcene, linalool and mixtures thereof.

In one embodiment, the hydrocarbon delivery device is designed to be located substantially externally of the housing of the air decontamination device, to avoid interference from the plasma cell, the UV radiation emitting device and/or the ozone catalysing device.

In another embodiment, as mentioned above, the hydrocarbon delivery device is designed to be located internally of the housing of the air decontamination device, and avoids interference from the plasma cell, the UV radiation emitting device and/or the ozone catalysing device by being located in the third air passage where it is protected by the shield.

It is noted that the hydrocarbon delivery device may alternatively be an aerosol or other pressurised container fitted near the air outlet of the air decontamination device.

Thus, the hydrocarbon delivery device may comprise a pressurised container containing one or more hydrocarbons distributed in an aqueous medium.

The presence of a hydrocarbon delivery device means that, should the UV radiation emitting device fail for any reason, the residual ozone levels in the treated air are within acceptable levels for human exposure.

Preferably the air decontamination device comprises an air stream generator which may be located at the air inlet of the housing, upstream of the plasma cell. This air stream generator may be spaced from the plasma cell; a stand or another spacing structure may be used to achieve this.

In an embodiment provided with a shield, the shield preferably has a length extending from at least the air stream generator to at least the end of the ozone catalysing device remote from the non-thermal plasma cell and/or to at least the end of the ultraviolet (UV) radiation emitting device remote from the non-thermal plasma cell.

The air decontamination device may also comprise an air turbulence creating device located within the housing at or towards the air outlet, downstream of the plasma cell.

Preferably the air stream generator is a fan. The turbulence creating device may also be a fan. The use of two fans (for example), one near the air inlet and one near the air outlet of the housing, assists in improving air flow and may reduce noise levels compared to the use of a large single fan at the air inlet.

In an alternative embodiment, the air turbulence creating device is not a fan but is a structure shaped and positioned to terminate at least a portion of the air flow passage of the housing with at least one curved or linear surface adjacent the air outlet of the housing, the surface being adapted to mix, and optionally re-direct, air passing through the device. Air flowing along the length of the housing may be re-directed transversely or obliquely, for example.

The air turbulence creating device may be used with the embodiment of the device having a first air passage and a second air passage; it may also be used with the embodiment of the device having first, second and third air passages.

The air turbulence creating device is preferably shaped to have a vertex located upstream of its base; the base may be circular, square, oval or rectangular in shape. The air turbulence creating device may be substantially cone-shaped, trumpet-shaped or pyramidal in shape. In addition, or alternatively, the air turbulence creating device may be spiral-shaped or have a twisted structure.

One effect of the air turbulence creating device is to mix the air streams coming from the respective air passages. Another possible effect is to prevent UV light from the UV radiation emitting device being visible externally of the device.

The present invention also provides a method of decontaminating air, preferably using the device of the present invention, the method comprising the steps of:
a) directing an air stream to be decontaminated through and across a non-thermal plasma cell so that free radicals are produced by which contaminants in the air stream are oxidised;
b) directing an air stream to be decontaminated externally of a non-thermal plasma cell so that free radicals are produced by which contaminants in the air stream are oxidised;
c) controlling ozone in the air stream output from the non-thermal plasma cell; and
d) introducing a hydrocarbon with two or more carbon-carbon double bonds into the air stream to react with residual ozone to control the air quality so that the air stream is suitable for human exposure.

The method may further comprise directing an air stream, which has not been decontaminated by the effects of a non-thermal plasma cell, to mix with and dilute the air streams decontaminated by the effects of the non-thermal plasma cell.

The plasma cell may operate continuously or at timed intervals. Operating the plasma cell at timed intervals produces pulses of treated air; such pulses of treated air, each producing a cascade of radicals, are effective in decontaminating air whilst using less energy.

The step of controlling ozone in the air stream output from the non-thermal plasma cell is preferably achieved by subjecting the air stream to UV radiation, and/or by exposing the air stream to a catalyst to accelerate the breakdown of ozone.

The present invention also provides an air decontamination device comprising a non-thermal plasma cell, an ultraviolet radiation emitting device, an ozone catalysing device, a hydrocarbon delivery device, and an air stream generator by which an air stream can be generated and directed to pass through and across the non-thermal plasma cell; wherein the plasma cell is sized and positioned within the air decontamination device such that some of the generated air stream can be directed through the air decontamination device without passing through and across the non-thermal plasma cell.

In one embodiment, the device further comprises at least one shield which is positioned such that some of the generated air stream is kept away from the non-thermal plasma cell and is shielded from the plasma of the non-thermal plasma cell.

The non-thermal plasma cell is preferably coincident with, partly coincident with or upstream of the UV radiation emitting device and the hydrocarbon delivery device has a hydrocarbon emitter which is preferably downstream of the UV radiation emitting device. The plasma cell may be upstream of the ozone catalysing device and the hydrocarbon emitter may be downstream of the ozone catalysing device.

The device and method of the present invention provide advantages over the prior art. By way of example, the device in operation creates less back-pressure, thereby requiring less energy to maintain an air-flow through the cell. This lowering of back-pressure also reduces noise levels.

Further, a greater volume of air can be passed through the device since, contrary to the prior art, it is not necessary to force all the incoming air through the plasma cell. This has the benefit that the concentration of unwanted by-products in the volume of air within the air decontamination device is lower (ie there is a dilution effect). In one example, the device of the present invention permits 24 m³/hr of air to pass therethrough with a 4 watt fan, compared to 18 m³/hr with a 16 watt fan in a previous device of the inventor.

It has also been found that the present invention results in the cascade reaction being more effective in the air in the chamber outside of the device. For example, the air passing through the air decontamination device is projected further into the chamber.

In addition, the hydrocarbon delivery device protects the hydrocarbon from premature oxidation such that the hydrocarbon is more effective at decontaminating the air. Positioning the air outlet of the hydrocarbon delivery device near the exit of the housing assists in the formation of a free cascade reaction which overspills into the chamber being treated.

Many devices exist to decontaminate air. The present invention differs in that the greater part of decontamination is carried out by reactive emissions from the device, ie in the open air of the chamber being treated.

The principle activity of the UV photocatalysis is the control of undesirable by-products, such as formaldehyde, produced by the activity of the plasma cell in normal operation. The UV photocatalysis does not contribute significantly to the overall decontamination of the chamber being treated.

The present invention will now be described, by way of example only, with reference to the accompanying diagrammatic drawings, in which:

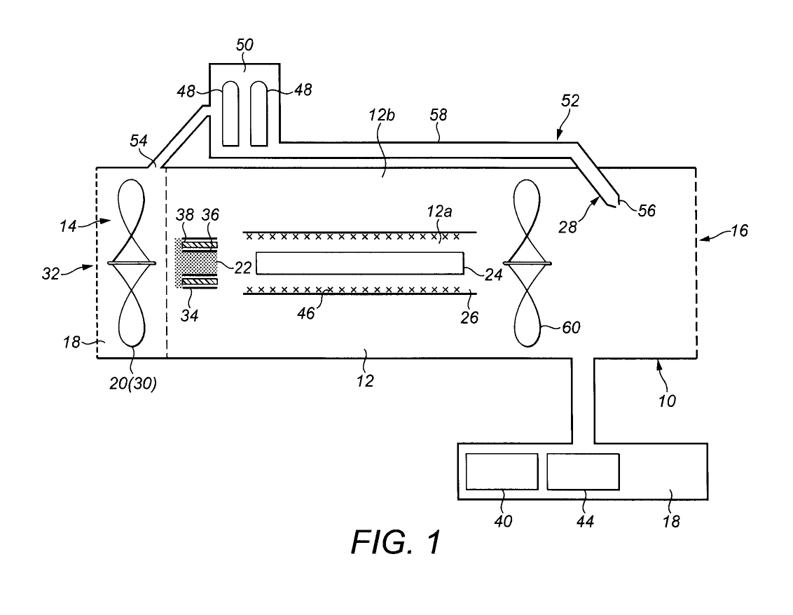

FIG. 1 shows a diagrammatic cross-sectional side view of an air decontamination device, in accordance with a first embodiment of the invention;

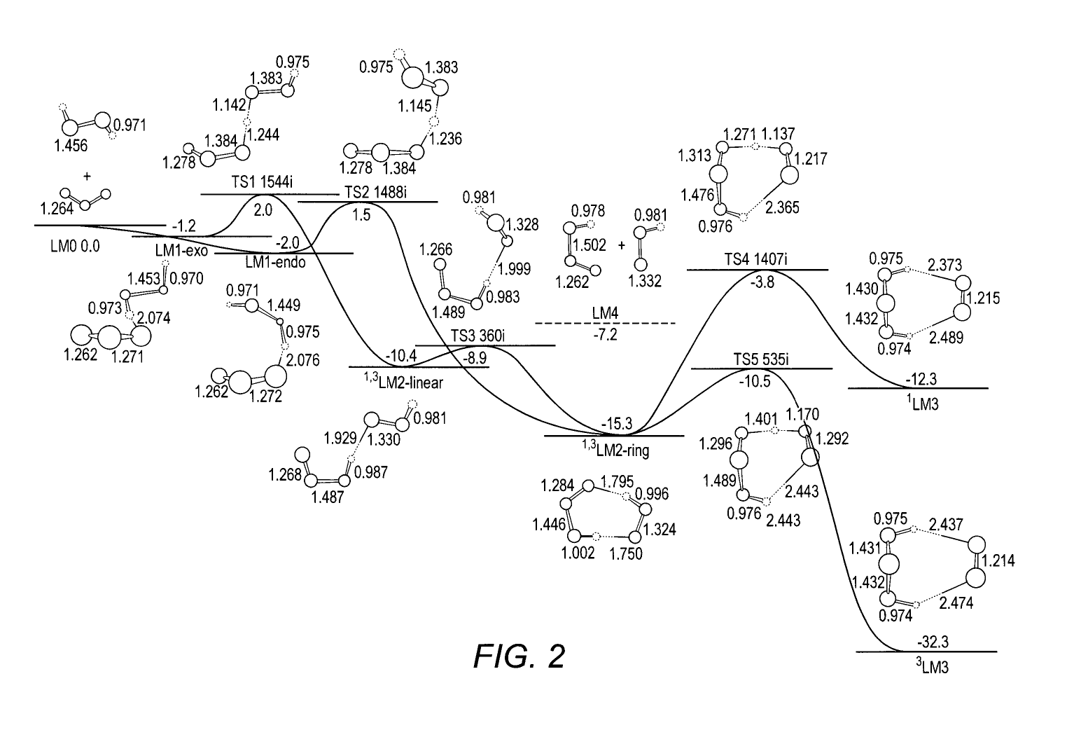

FIG. 2 shows perozone chemistry;

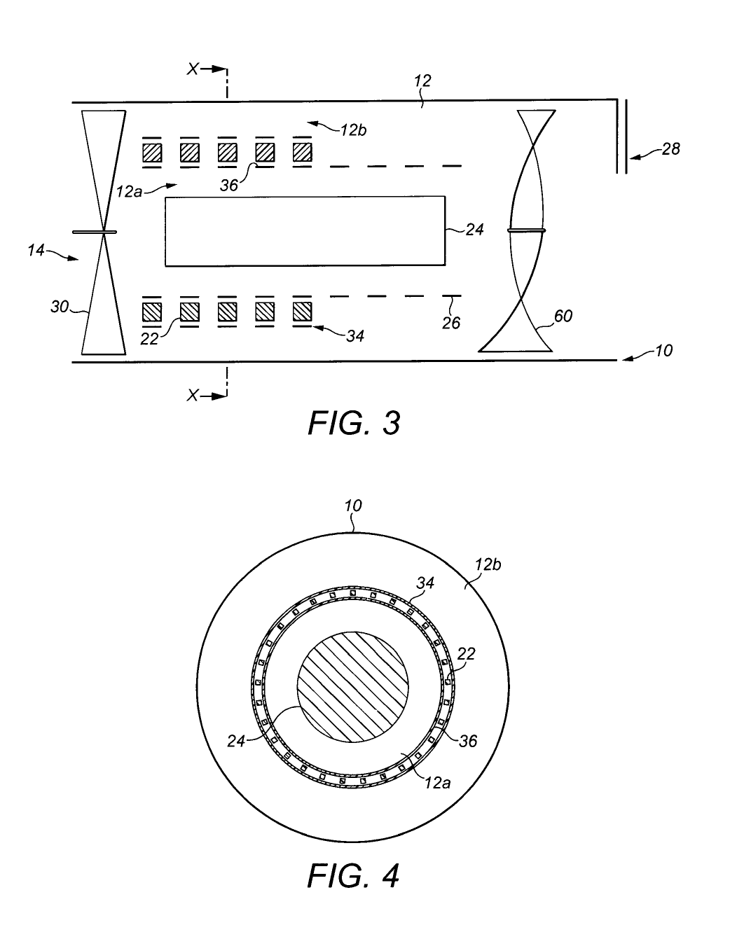

FIG. 3 shows a diagrammatic cross-sectional side view of an air decontamination device, in accordance with a second embodiment of the invention;

FIG. 4 shows a cross-sectional view along line X-X of FIG. 3;

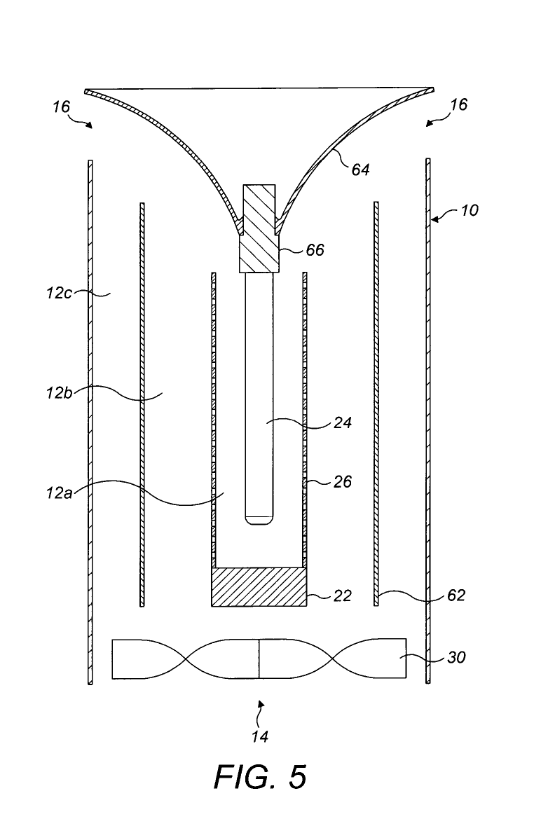

FIG. 5 shows a diagrammatic cross-sectional side view of an air decontamination device, in accordance with a third embodiment of the invention; and

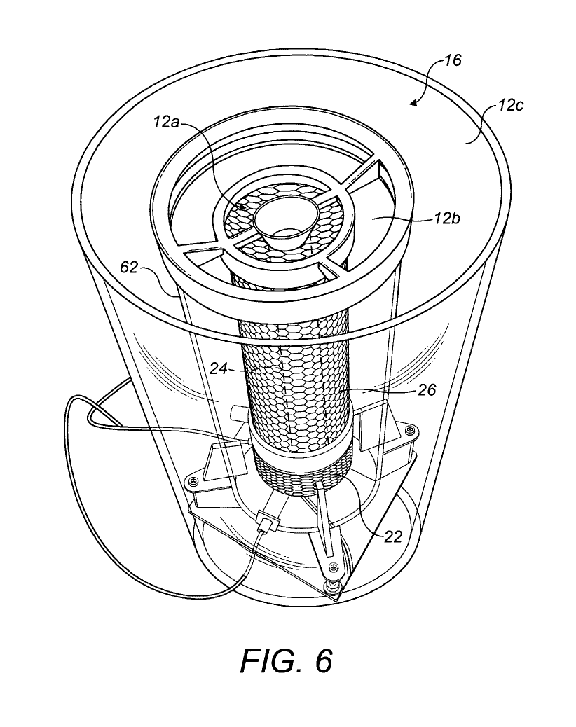

FIG. 6 shows a perspective view of part of the air decontamination device according to the third embodiment of the invention.

Referring to FIG. 1, there is shown an air decontamination device which comprises a housing 10 having a flow passage 12, an air inlet 14 to the flow passage 12 and an air outlet 16 exiting from the flow passage 12, and a compartment 18 adjacent to the flow passage 12. An air stream generator 20, a non-thermal plasma cell 22, an ultraviolet (UV) radiation emitting device 24, an ozone catalysing device 26, and a hydrocarbon emitter 28 are located in the passage 12. The emitter forms part of a hydrocarbon delivery device 52. The flow passage 12 has a first (inner) passage 12a and a second (outer) passage 12b.

The air stream generator 20 is provided adjacent the air inlet 14 of the passage 12. The air stream generator 20, in this embodiment, is an electric fan 30 powered by mains electricity or battery packs (not shown) provided in the compartment 18 of the housing 10. As a safety measure, a grill 32 is provided across the air inlet 14 to prevent accidental access to the fan 30 while in operation.

The non-thermal plasma cell 22 is positioned adjacent the fan 30, downstream of the air inlet 14. In one embodiment the plasma cell 22 comprises an annular ceramic dielectric ring having a series of circular holes or longitudinal slots around its circumference. Sheets of metal electrodes, forming a cathode 34 and an anode 36, are wrapped around its circumference. Thus, preferably, the dielectric is a perforated ring of ceramic material, with porous annular electrodes internally and externally fitted, about the circumference. The cathode 34 and anode 36 are powered by an adjustable power supply unit (PSU) 40 housed in the compartment 18 of the housing 10.

The cathode 34 and anode 36 comprise reticulated (three dimensionally porous) conductive elements, in this case being ceramic and stainless steel composites. However, any rigid reticulated conductive or semi-conductive material could be used.

The dielectric 38 may be ceramic. However, again, the dielectric 38 could be any suitable material to suit varying applications and specific requirements. The dielectric 38 material may be coated with a catalytic material.

The UV radiation emitting device 24 includes an ultraviolet light emitting device powered by a PSU 44 (power supply unit) housed in the compartment 18 of the housing 10. The UV light emitting device is disposed in the passage 12, downstream of the non-thermal plasma filter 22, and coincident with the ozone catalysing device 26. The UV light emitting device may comprise one or more UV tubes or UV LEDs.

The ozone catalysing device 26 comprises a mesh 46 disposed across the passage 12 and surrounding the UV radiation emitting device 24. The mesh 46 includes a coating of ozone catalysing material, such as a mixture of titanium, lead and manganese oxides.

The hydrocarbon delivery device 52 comprises a hydrocarbon emitter 28 which is supplied from a rechargeable hydrocarbon reservoir 48 located in an evaporator chamber 50 for evaporating liquid hydrocarbon held in the reservoir 48. The liquid hydrocarbon is preferably contained within a membrane. The hydrocarbon delivery device has an air inlet 54, an air outlet 56 and an air flow passage 58 therebetween. The air flow passage is located, at least in part, outside of the housing of the air decontamination device. The hydrocarbon reservoir and the evaporator chamber are disposed along the air flow passage so that air passing through the air flow passage picks up discharged gaseous hydrocarbon and delivers it to the hydrocarbon emitter 28. The air stream generator (fan 30) assists in driving the hydrocarbon-containing air through the hydrocarbon delivery device.

In this embodiment, the air flow passage of the hydrocarbon delivery device is only in fluid communication with the air flow passage of the air decontamination device at the air inlet and air outlet of the hydrocarbon delivery device. This has the advantage that air entering the air flow passage 58 from the air inlet 54 is substantially free of ozone produced by the plasma cell, meaning that the hydrocarbon picked up by the flowing air is not immediately oxidized by ozone and other by-products. Hence, when released by the hydrocarbon emitter, the hydrocarbon is effective in reacting with and controlling residual ozone in the air stream about to exit the air decontamination device.

The air inlet of the hydrocarbon delivery device is preferably adapted to draw air from the vicinity of the air inlet of the housing of the air decontamination device. The air flow produced by the air stream generator creates a pressure differential between the air inlet (which has a positive pressure) and the air outlet (which has a negative pressure) of the hydrocarbon delivery device. This pressure differential acts to drive the hydrocarbon through the hydrocarbon delivery device, resulting in a sufficiently constant delivery of the hydrocarbon through the hydrocarbon emitter.

The hydrocarbon reservoir 48 contains a liquid hydrocarbon, for example an olefin such as a terpene and, more specifically, myrcene or linalool.

The outlet of the hydrocarbon emitter 28 is located at or in the vicinity of the centre of the passage 12 of the housing 10, and downstream of the UV light emitting device and mesh 46 of the ozone catalysing device 26. The outlet of the hydrocarbon emitter 28 is therefore located adjacent the outlet 16 of the passage 12 of the housing 10.

Preferably, the rate of hydrocarbon emission is matched to the output of ozone produced by the air decontamination device, such that, in an equilibrium state, minimal surplus ozone (5-45 ppb) is reacted with minimal hydrocarbon to achieve a hydroxyl radical measurement of not less than 10⁶/cc.

Any other suitable means for supplying volatilised hydrocarbon to the outlet of the hydrocarbon emitter 28 can be used, provided that the hydrocarbon source is provided with an air supply not affected by the plasma cell or UV catalysis.

For example, the hydrocarbon delivery device may alternatively be an aerosol or other pressurised container fitted near the air outlet of the air decontamination device.

In this respect, the hydrocarbon, such as a terpene (for example, linalool), may be blended with an agent such as a surfactant or a chemical with properties which will render the terpene miscible with water, but will not react with the terpene, and has no properties which compromise the performance or safety of the device. There are many suitable surfactants commercially available which conform to these requirements. The hydrocarbon is then mixed with water which is preferably degassed and de-ionised.

The resulting hydrocarbon in an aqueous medium will enable the use of pressurised dispensers such as aerosol containers. Such pressurised dispensers may have outlets designed to work with the electronic controls of the air decontamination device for accurate dosing with very small amounts of terpene diluted in water. The very small amounts of terpene required for effectiveness, typically <50 mg/day, are difficult to control with a simple evaporative technique, such as used in the earlier devices. The dispensing container may be pressurised with an inert gas which will prevent degradation of the terpene, as will the degassing of the de-ionised water.

The hydrocarbon mixture may be sprayed from a suitably configured spray-head or other system consistent with highly accurate dosing to produce micro-droplets of water typically less than 0.5 microns.

De-ionised water is used to avoid reaction with the terpene or corrosion of the storage and delivery system, but has the advantage of humidifying the emissions from the device which will enhance hydroxyl radical output.

This is achieved because the terpene and surfactant mixture slightly reduces the surface tension of the water droplets in the spray. This has the effect of increasing the solubility of gaseous ozone in the water droplets. The ozone is naturally only sparingly soluble in water. When ozone gas surrounds a water micro-droplet, molecules of ozone move through the surface tension of the droplet which acts as a membrane, to react with either water molecules or terpene. With water, the ozone will produce peroxone, H₂O₃, which is highly reactive and then hydrogen peroxide; the reaction of ozone with the terpenes produces hydroxyl and hydroperoxyl radicals which in turn react with the hydrogen peroxide to produce more hydroxyl radicals. The water molecules released with this reaction also provide a source of hydrogen and oxygen atoms for further production of hydroxyl and hydroperoxyl radicals.

FIG. 2 illustrates peroxone chemistry, including the formation of H₂O₃ and ring-(HO₂)(HO₃) from O₃.

Using a pressurised container, such as an aerosol, can simplify the hydrocarbon delivery process for certain applications but is not universally appropriate, particularly where devices remain isolated for extended periods.

At the air outlet 16 of the housing there is optionally provided a second fan 60 or another means of creating air turbulence, such as an obstruction (eg a non-motorised rotary blade). This is to provide better mixing of the air which has passed through the housing of the air decontamination device and the air which is delivering the hydrocarbon, resulting in decontaminated air with acceptable levels of ozone and formaldehyde (for example).

The air decontamination device can be solely powered by mains electricity, solely powered by battery packs, which may be rechargeable, or may be selectively energisable by both power sources.

The air decontamination device can be produced in the form of a portable device. Alternatively, the air decontamination device can be produced as a larger device intended to remain in one location once installed. The latter device is more suitable for, but not limited to, industrial or commercial installations and premises.

In use, the air decontamination device is positioned in the location to be decontaminated. The device is intended to decontaminate air within a building, chamber, enclosure, trunking, pipe, channel or other enclosed or substantially enclosed area. The device should be regulated to suit the chamber size being treated, so that the ozone level at equilibrium should never rise above pre-set levels under any circumstances due to failure of other components.

The device is preferably designed to emit not less than 10⁶ hydroxyl radicals per cc air.

In use, the device is energised, and the fan 30 generates a stream of ambient air along the first passage 12a and the second passage 12b of the housing 10. The air stream in the first passage passes initially through the non-thermal plasma cell 22. The air stream in the second passage passes initially outside of the external surface of the non-thermal plasma cell 22.

The plasma cell utilises the characteristics of a non-thermal plasma to ‘plasmalise’ the constituent parts of the air. In general terms, the outer ring electrons in the atomic structure of the elements comprising air (principally oxygen and nitrogen) are ‘excited’ by the intense electronic field generated by the non-thermal plasma, typically being 10 Kv at 20-30 KHz. The energised electrons release energy through collisions. However, little or no heat is emitted due to the insubstantial mass of the electrons and the consequent lack of ionisation that occurs. The release energy is sufficient to generate radicals within the air stream, such as O. and OH.. The radicals are powerful oxidants, and will oxidise hydrocarbons, organic gases, and particles typically 2.5 μm and below, such as bacteria, viruses, spores, yeast moulds and odours and carbon particles. Only the most inert elements or compounds will generally resist oxidation.

Since many of the resultants of the oxidative reactions are transient and surface acting, due to having zero vapour pressure, by providing a molecular thick catalytic coating on some or all of the dielectric material of the non-thermal plasma, oxidation of particular molecules or compounds, for example nerve gas agents, within the non-thermal plasma can be targeted.

The non-thermal plasma cell 22 produces ozone as one of the by-products. This is entrained in the air stream leaving the non-thermal plasma cell 22. The half-life of ozone is dependent on atmospheric conditions and, itself being a powerful oxidant, under normal circumstances will continue to react in the air long after it has exited the plasma core. The initial control of excess ozone is carried out by the specification of the plasma cell power supply 40, by regulating the input of volts/current and by regulating the input of air by controlling the air stream generator 20.

The airstream leaving the non-thermal plasma cell 22 in the first passage passes over the UV radiation emitting device 24 within the surrounding ozone catalysing device 26. The airstream leaving the vicinity of the non-thermal plasma cell 22 in the second passage passes over the external surface of the ozone catalysing device 26. Although not shown in the diagrammatic drawing of FIG. 1, the ozone catalysing device is preferably close to or in contact with the non-thermal plasma cell to assist with the formation of the first and second passages; in this respect, a bridging piece (such as the ring-shaped piece shown in FIG. 6) may be positioned between the ozone catalysing device and the non-thermal plasma cell to assist with the formation of the first and second passages. The first passage and the second passage are permeable so that flowing air can permeate between them, meaning that the airstream in the second passage is also subject to the effects of the UV radiation emitting device and the ozone catalysing device.

Ultraviolet radiation emitted at 253.4-378 nanometres wavelength by the UV light emitting device acts to break down some of the ozone entrained in the air stream. The coating on the mesh 46 acts to catalyse this break down.

The destruction (photo-oxidation) of the ozone increases the radical level, particularly the level of hydroxyl radicals OH., within the air stream. These radials also vigorously oxidise contaminants remaining within the air stream.

The secondary fan 60 provides energy and turbulence to the treated air prior to it reaching the hydrocarbon emitter 28. This is to ensure good mixing of the air.

Trials have shown that hydroxyl and other radicals resident in the air stream after ‘plasmalising’ significantly increase the rate of generation of free radicals during the photo-oxidative process.

It is not desirable to destroy all of the ozone entrained in the air stream from the plasma cell 22 using the UV radiation emitting device 24 and the ozone catalysing device 26.

The air stream in the first passage and the second passage exits the area of the ozone catalysing device 26, is mixed by the secondary fan 60 and passes along the passage 12 to the hydrocarbon emitter 28. The hydrocarbon emitter 28 discharges volatilised hydrocarbon into the air stream in order to control the remaining residual ozone to achieve desirable levels. Myrcene is suggested, since it is naturally occurring, has no known toxicity, and is widely used to ‘extend’ perfumes and fragrances. However, linalool is preferred.

Linalool contains two carbon-carbon double bonds in its molecular structure:

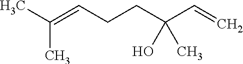

Ozone reacts preferentially with linalool evaporated into the air stream. When linalool reacts with ozone, a ‘free radical cascade’ is triggered. More than thirty interrelated reactions occur, many of which produce a series of short half-life oxidants such as hydro peroxides, super oxides, hydroxy peroxides, and hydroxyl peroxides. Each of these oxidants breaks down, releasing yet further free radicals, which in turn promulgate the production of these oxidative species. This process continues in the chamber/area being treated until an equilibrium is reached between the emission and destruction of ozone by its reaction with the carbon-carbon double bonds of the hydrocarbon.

The products of these preferential reactions have zero vapour pressure, and hence condense on any remaining particle in the air stream or surface. As a result, decontamination of contaminants within the ambient air occurs, once the decontaminated air stream exits through the outlet 16 of the housing 10.

Due to the air decontamination device effectively recirculating and re-decontaminating air within an environment (eg the chamber/area), small particulates are removed as a result of the use of the non-thermal plasma cell 22, such that the device has an air filtering effect.

The air stream generator can be driven in reverse, enabling decontamination of the interior of the device by drawing excess free radicals entrained in the air stream back through the device. As such, the device is largely self-cleaning.

Referring to FIGS. 3 and 4, in a second embodiment of the invention, an air decontamination device comprises a housing 10 having a flow passage 12, an air inlet 14 to the flow passage 12 and an air outlet exiting from the flow passage 12. The device also has a compartment and a hydrocarbon delivery device in accordance with the first embodiment but these are not shown in FIG. 3 or 4. An air stream generator (eg fan 30), a non-thermal plasma cell 22, an ultraviolet (UV) radiation emitting device 24, an ozone catalysing device 26, and a hydrocarbon emitter 28 are located in the passage 12. The flow passage 12 has a first (inner) passage 12a and a second (outer) passage 12b.

Unless stated otherwise, the second embodiment of the device operates in the same way as the first embodiment of the device.

In this second embodiment, the UV radiation emitting device includes an ultraviolet light emitting tube which is disposed at least in part within the central region of an annular ring of the non-thermal plasma cell. The plasma field within this annular ring may be used to excite mercury provided in a mercury vapour tube to emit the UV radiation, in addition to decontamination of the air. This means that a separate power source for the UV radiation emitting device is not required.

Referring to FIG. 5, in a third embodiment of the invention, an air decontamination device comprises a housing 10 having a flow passage 12, an air inlet 14 to the flow passage 12 and an air outlet 16 exiting from the flow passage 12.

Unless stated otherwise, the third embodiment of the device operates in the same way as the first embodiment of the device. Although the ultraviolet (UV) radiation emitting device of the third embodiment is not positioned within the non-thermal plasma cell, it is possible for the ultraviolet (UV) radiation emitting device of the third embodiment to be positioned within the non-thermal plasma cell, in accordance with the second embodiment.

An air stream generator (eg fan 30), a non-thermal plasma cell 22, an ultraviolet (UV) radiation emitting device 24, an ozone catalysing device 26, and a hydrocarbon delivery device are located in the flow passage 12. The flow passage 12 has a first (inner) passage 12a, a second (middle) passage 12b and a third (outer) passage 12c.

The air stream generator is preferably larger in diameter than in the first embodiment to enable air to flow into all three passages. The air stream generator is spaced from the non-thermal plasma cell in the longitudinal direction of the housing.

A shield 62 is provided between the second (middle) passage 12b and the third (outer) passage 12c.

The shield 62 extends in the direction of the flow passage, the shield being positioned between, and spaced from, the non-thermal plasma cell 22 and the wall or walls of the housing 10.

As a result, a portion of air entering the housing from the air inlet 14 is adapted to pass outside of the external surface of the shield via third passage 12c, this portion of air being separate from the portion of air which is adapted to pass through and across the non-thermal plasma cell via first passage 12a, and being separate from the portion of air which is adapted to pass outside of the external surface of the non-thermal plasma cell via second passage 12b.

The different portions of air flowing towards the exit of the housing at the air outlet 16, are adapted to be mixed together at or adjacent to the air outlet to provide a dilution effect: in this respect, the untreated air from the third passage dilutes the treated (decontaminated) air from the first and second passages.

In this embodiment, the internal surface of the third passage 12c is defined by the external surface of the shield 62, and the external surface of the third passage is defined by the internal surface of the housing 10. Also, the internal surface of the second 12b passage is defined by the external surface of the non-thermal plasma cell 22 and the external surface of the ozone catalysing device 26, and the external surface of the second passage 12b is defined by the internal surface of the shield 62.

The housing is elongate and the first, second and third passages extend in the direction of the length of the housing, with the first passage 12a being surrounded by the second passage 12b and with the second passage being surrounded by the third passage 12c.

The shield is impermeable to air to prevent air moving from either the first passage 12a or the second passage 12b into the third air passage 12c.

Preferably the shield is made of metal or metalised plastic. It may be cylindrical in shape. It preferably has a length extending from the region of the air stream generator to at least the end of the ozone catalysing device remote from the non-thermal plasma cell and/or to at least the end of the ultraviolet (UV) radiation emitting device remote from the non-thermal plasma cell.

The shield is adapted to shield air flowing in the third passage 12c from electromagnetic emissions from the non-thermal plasma cell. It may also provide a light-proof barrier to the UV rays from the ultraviolet (UV) radiation emitting device 24.

The internal surface of the shield may be coated with a catalyst, for example titanium dioxide, to further enhance the breakdown of excess ozone, and to utilise incident UV light to produce a greater yield of hydroxyl radicals.

The shield may be used to form an electrostatic surface, able to act as an electrostatic decontamination device for the deposition of particles in the first and second air passages charged by proximity to the plasma cell. In this respect, the shield may be earthed.

A hydrocarbon delivery device is located in the third passage 12c to provide hydrocarbons to the hydrocarbon emitter using the force of the untreated air stream flowing through the third passage or by using a syphoning effect, for example.

The flow passage 12, at the air outlet 16, is terminated (at least in part) by an air turbulence creating device 64. This air turbulence creating device may be cone-shaped or trumpet-shaped: it has a vertex located upstream of its base which may be circular, square or rectangular in shape.

The effect of the air turbulence creating device is to mix the air streams coming from the first, second and third passages, and also to change the direction of these air streams such that the air may be emitted obliquely from the device, if desired. The air turbulence creating device may also be used with embodiments of the invention which do not have the third passage.

The air turbulence creating device 64 is preferably attached to the ultraviolet (UV) radiation emitting device 24 by a cap 66. The air turbulence creating device 64 may be designed and positioned such that it prevents the UV light from being externally visible by users of the device.

Referring to FIG. 6, the air decontamination device comprises the housing having flow passage 12, the air inlet to the flow passage 12 and the air outlet 16 exiting from the flow passage 12. Please note that some features have been omitted from FIG. 6 for clarity.

The air stream generator, the non-thermal plasma cell 22, the ultraviolet (UV) radiation emitting device 24, and the ozone catalysing device 26 are located in the flow passage 12. The flow passage 12 has the first (inner) passage 12a, the second (middle) passage 12b and the third (outer) passage 12c. The external surface of the first (inner) passage 12a and the internal surface of the second (middle) passage 12b are defined by the external surface of the non-thermal plasma cell and the external surface of the ozone catalysing device; a ring-shaped bridging piece (by way of example) is positioned between the non-thermal plasma cell and the ozone catalysing device to assist with the formation of the first (inner) and second (middle) passages. Shield 62 is provided between the second (middle) passage 12b and the third (outer) passage 12c.

The embodiments described above are given by way of example only, and modifications will be apparent to persons skilled in the art without departing from the scope of the invention as defined by the appended claims.

1. A device for air decontamination comprising a housing having an air inlet, an air outlet and an air flow passage therebetween, the housing including at least one non-thermal plasma cell located downstream of the air inlet; wherein the non-thermal plasma cell is sized and positioned relative to the internal dimensions of the housing such that a portion of air entering the housing from the air inlet is adapted to pass through and across the non-thermal plasma cell and a portion of air entering the housing from the air inlet is adapted to pass outside of the external surface of the non-thermal plasma cell.

2. A device for air decontamination as claimed in claim 1, further comprising at least one UV radiation emitting device and/or at least one ozone catalysing device which are located within the housing, coincident with, partly coincident with or downstream of the non-thermal plasma cell.

3. A device for air decontamination as claimed in claim 1, wherein the air flow passage in the housing of the air decontamination device comprises a first air passage and a second air passage, the first air passage being adapted to carry the portion of air which passes through and across the non-thermal plasma cell and the second air passage being adapted to carry the portion of air which passes outside of the external surface of the non-thermal plasma cell.

4. A device for air decontamination as claimed in claim 3, wherein the first air passage and the second air passage are permeable to permit air to move therebetween.

5. A device for air decontamination as claimed in claim 3, wherein the UV radiation emitting device is positioned within the first air passage of the housing.

6. A device for air decontamination as claimed in claim 3, wherein the ozone catalysing device is positioned such that air in the first air passage is adapted to pass through the ozone catalysing device and air in the second air passage is adapted to pass over the external surface of the ozone catalysing device.

7. A device for air decontamination as claimed in claim 3, wherein the external surface of the first air passage is defined by the external surface of the non-thermal plasma cell and the external surface of the ozone catalysing device.

8. A device for air decontamination as claimed in claim 3, wherein the housing is elongate and the first and second air passages extend in the direction of the length of the housing, with the first air passage being surrounded by the second air passage.

9. A device for air decontamination as claimed in claim 1, further comprising a device for delivering hydrocarbons in the vicinity of the air outlet of the housing of the air decontamination device, said hydrocarbon delivery device having an air inlet, an air outlet and an air flow passage therebetween.

10. A device for air decontamination as claimed in claim 9, wherein the hydrocarbon delivery device comprises a reservoir of one or more hydrocarbons located in the air flow passage of the hydrocarbon delivery device, such that incoming air is adapted to pick up the hydrocarbons as it flows through the air flow passage, and wherein the hydrocarbons in the air are delivered within the housing of the air decontamination device in the vicinity of the air outlet of the housing.

11. (canceled)

12. (canceled)

13. A device for air decontamination as claimed in claim 1, wherein the housing further comprises a shield extending in the direction of the air flow passage, the internal surface of the shield being spaced from and facing the non-thermal plasma cell, such that a portion of air entering the housing from the air inlet is adapted to pass outside of the external surface of the shield, this portion of air being separate from the portion of air which is adapted to pass through and across the non-thermal plasma cell and being separate from the portion of air which is adapted to pass outside of the external surface of the non-thermal plasma cell.

14. A device for air decontamination as claimed in claim 13, wherein the housing further comprises a third air passage which is adapted to carry the portion of air which passes outside of the external surface of the shield.

15. A device for air decontamination as claimed in claim 14, wherein the shield is adapted to shield air flowing in the third air passage from electromagnetic emissions from the non-thermal plasma cell.

16. A device for air decontamination as claimed in claim 14, wherein the housing is elongate and the first, second and third air passages extend in the direction of the length of the housing, with the first air passage being surrounded by the second air passage and with the second air passage being surrounded by the third air passage.

17. A device for air decontamination as claimed in claim 1, further comprising an air stream generator located at the air inlet of the housing, upstream of the non-thermal plasma cell.

18. A device for air decontamination as claimed in claim 1, further comprising an air turbulence creating device located within the housing at or towards the air outlet of the housing, downstream of the non-thermal plasma cell.

19. A device for air decontamination as claimed in claim 18, wherein the air turbulence creating device is shaped and positioned to terminate at least a portion of the air flow passage of the housing with at least one curved or linear surface adjacent the air outlet of the housing, the surface being adapted to mix, and optionally re-direct, air passing through the device.

20. A method of decontaminating air comprising the steps of:
a) directing an air stream to be decontaminated through and across a non-thermal plasma cell so that free radicals are produced by which contaminants in the air stream are oxidised;
b) directing an air stream to be decontaminated externally of the non-thermal plasma cell so that free radicals are produced by which contaminants in the air stream are oxidised;
c) controlling ozone in the air streams output from the non-thermal plasma cell; and
d) introducing a hydrocarbon with two or more carbon-carbon double bonds into the air stream to react with residual ozone to control the air quality so that the air stream is suitable for human exposure.

21. A method of decontaminating air as claimed in claim 20, wherein the method further comprises directing an air stream, which has not been decontaminated by the effects of a non-thermal plasma cell, to mix with and dilute the air streams decontaminated by the effects of the non-thermal plasma cell.

22. A method of decontaminating air as claimed in claim 20 using the device claimed in claim 1.
